(19) United States
(12) Patent Application Publication
(10) Pub. No.: US 2010/0008993 A1
(43) Pub. Date: Jan. 14, 2010

(54) Compositions and Methods for Increasing Bioavailability of Topical Ophthalmic Drugs

(76) Inventors: Joel W. Proksch, Pittsford, NY (US); Keith W. Ward, Ontario, NY (US)

Correspondence Address: Bausch & Lomb Incorporated, One Bausch & Lomb Place, Rochester, NY, 14604-2701, US

(21) Appl. No.: 12/172,325

(22) Filed: Jul. 14, 2008

(51) Int. Cl. A61K 9/12; A61K 31/4709; A61P 27/00

(52) U.S. Cl. 424/489; 514/313

(57) ABSTRACT

An ophthalmic composition comprises an ophthalmic drug that has a low solubility in water and a surfactant, wherein the ophthalmic drug is present at a concentration from about 3 to about 7000 times the solubility of the drug in water. A volume of about 1-15 microliter is administered topically to an eye of a subject to treat or control a condition for which the drug is effective.

### BACKGROUND

The present invention relates to compositions and methods for increasing the bioavailability of topical ophthalmic drugs. In particular, the present invention relates to compositions and methods for increasing the bioavailability of topical ophthalmic drugs having low solubility in water.

Topical ophthalmic compositions have taken the form of liquids (including solutions and suspensions), ointments, gels, and inserts. Liquid compositions for drop-wise instillation of pharmaceutically active agents to the eye provide for easy administration, but they do not always provide for an accurate desired dosage amount in the relevant ocular tissues, as portions of the liquid are often blinked away during administration, drained through the punctum into the nasal passage, or diluted by high tear fluid turnover. After instillation of an eye-drop, typically less than 5% of the applied drug penetrates the cornea and reaches intraocular tissues. After topical administration of an ophthalmic drug composition, the drug is first diluted by the lacrimal fluid. A large portion of the dose is lost to outside the eye through overflow. The contact time of drug remaining in the eye with ocular tissues is relatively short (about 1 to 2 minutes) because of the continuous production of lacrimal fluid (about 0.5 to 2.2 μL/minute). Then, approximately half of the drug remaining on the eye flows through the upper canaliculus and the other half, through the lower canaliculus into the lacrimal sac, which opens into the nasolacrimal duct. Drainage of lacrimal fluid during blinking (every 12 seconds) towards the nasolacrimal duct induces a rapid elimination of the dose. On the other hand, ointments and gels, which usually reside in the eye longer than a liquid and therefore provide for larger retention of the drug, often interfere with a patient's vision. Ocular inserts, both bioerodible and non-bioerodible, are also available and allow for less frequent administration of drug. These inserts, however, require complex and detailed preparation and are frequently uncomfortable to the wearer. An additional problem with non-bioerodible inserts is that they must be removed after use. Thus, eye drops are still the preferred ophthalmic compositions because they are easily self-administered.

However, because of the aforementioned limitations imposed by the physiology of the eye, there is a continued need to provide improved methods for administering topical ophthalmic compositions. Improved topical ophthalmic compositions that can overcome these limitations are also desirable.

### SUMMARY

In general, the present invention provides a composition and a method for increasing the |bioavailabilty| of an ophthalmic drug in an ocular tissue of a subject.

In one aspect, the present invention provides a composition and a method for increasing the bioavailabilty of an ophthalmic drug that has a low solubility in water, in an ocular tissue of a subject.

In another aspect, a composition of the present invention comprises an ophthalmic drug that has a low solubility in water and a surfactant, wherein the ophthalmic drug is present at a concentration from about 3 to about 7000 times the solubility of said drug in water, said solubility being measured at about 25° C. and at pH of about 7-7.5.

In still another aspect, said composition comprises an aqueous suspension.

In yet another aspect, the present invention provides a method for increasing bioavailability of an ophthalmic drug in an ocular tissue of a subject. The method comprises administering topically to an ocular surface of said subject a volume from about 1 to about 15 microliter (“μL”) of a composition that comprises: (a) an ophthalmic drug that has a low solubility in water; and (b) a surfactant, wherein the ophthalmic drug is present at a concentration from about 3 to about 7000 times the solubility of said drug in water, said solubility being measured at about 25° C. and at pH of about 7-7.5, and wherein an amount of said drug is non-toxic to said subject.

In another aspect, said solubility is measured at about 25° C. and at pH of about 7.4.

In a further aspect, a composition or a method of the present invention can advantageously offer minimal interference with vision or disruption of the natural tear film.

Other features and advantages of the present invention will become apparent from the following detailed description and claims.

### DETAILED DESCRIPTION

As used herein, the phrase “low aqueous solubility” or “low solubility in water” means solubility in water of less than, or equal to, about 0.1 mg/mL at pH of about 7-7.5 and at about 25° C. Although compositions and methods of the present invention are particularly applicable to pharmaceutical components or compounds having such solubility, such compositions and methods are also useful in providing novel formulations of enhanced bioavailability of pharmaceutical compounds, which have solubility in water in the range of less than, or equal to, about 0.2 mg/mL (or, alternatively, less than, or equal to, about 0.5 mg/mL) and are difficult to be formulated into compositions having therapeutically significant concentrations of dissolved pharmaceutical components or compounds.

In general, the present invention provides a composition and a method for increasing the bioavailabilty of an ophthalmic drug in an ocular tissue of a subject.

In one aspect, the present invention provides a composition and a method for increasing the bioavailabilty of an ophthalmic drug that has a low solubility in water, in an ocular tissue of a subject.

In another aspect, the present invention provides a composition and a method for increasing the bioavailabilty of an ophthalmic drug that has solubility in water in the range from about 0.0001 to about 0.1 mg/mL (or, alternatively from about 0.0001 to about 0.07 mg/mL), in an ocular tissue of a subject, wherein the solubility is measured at pH of about 7-7.5 and at a temperature of about 25° C.

In still another aspect, the present invention provides a composition and a method for increasing the bioavailabilty of an ophthalmic drug that has solubility in water in the range from about 0.0001 to about 0.05 mg/mL (or alternatively, from about 0.0001 to about 0.03, or from about 0.0001 to about 0.02, or from about 0.001 to about 0.03, or from about 0.001 to about 0.02 mg/mL, or from about 0.0001 to about 0.2 mg/mL, or from about 0.0001 to about 0.5 mg/mL), in an ocular tissue of a subject, wherein the solubility is measured at pH of about 7-7.5 and at a temperature of about 25° C.

In yet another aspect, the solubility is measured at a pH of about 7.4 and at a temperature of about 25° C.

In a further aspect, a composition of the present invention comprises: (a) an ophthalmic drug that has a low solubility in water; and (b) a surfactant, wherein the ophthalmic drug is present at a concentration from about 3 to about 7000 times a solubility of said drug in water, said solubility being measured at about 25° C. and at pH of about 7-7.5, and wherein an amount of said drug is non-toxic to a subject in whom said composition is administered.

In some embodiments, the ophthalmic drug is present at a concentration from about 10 to about 5000 times a solubility of said drug in water. In some other embodiments, the ophthalmic drug is present at a concentration from about 10 to about 3000 times (or alternatively, from about 10 to about 2000, or from about 10 to about 1000, or from about 10 to about 500, or from about 10 to about 100, or from about 5 to about 100, or from about 5 to about 50, or from about 50 to about 2000, or from about 50 to about 1000, or from about 50 to about 100, or from about 100 to about 2000, or from about 100 to about 1000 times) a solubility of said drug in water.

In a further aspect, a composition of the present invention comprises: (a) an ophthalmic drug that has a solubility in water in a range from about 0.0001 to about 0.2 mg/mL (or, alternatively, from about 0.0001 to about 0.5 mg/mL); and (b) a surfactant, wherein the ophthalmic drug is present at a concentration from about 3 to about 2000 (or, alternatively, from about 3 to about 1000, or from about 10 to about 100) times a solubility of said drug in water, said solubility being measured at about 25° C. and at pH of about 7-7.5, and wherein an amount of said drug is non-toxic to a subject in whom said composition is administered.

In yet another aspect, an ophthalmic drug included in a composition of the present invention is selected from the group consisting of anti-inflammatory agents, anti-infective agents (including antibacterial, antifungal, antiviral, antiprotozoal agents), anti-allergic agents, antihistamines, antiproliferative agents, anti-angiogenic agents, anti-oxidants, antihypertensive agents, neuroprotective agents, cell receptor agonists, cell receptor antagonists, immunomodulating agents, immunosuppressive agents, intraocular (“IOP”) lowering agents, α₂-adrenergic receptor agonists, β₁-adrenergic receptor antagonists, carbonic anhydrase inhibitors, cholinesterase inhibitor miotics, prostaglandins and prostaglandin receptor agonists, mast cell degranulation inhibitors (mast cell stabilizers), thromboxane A₂ mimetics, protein kinase inhibitors, prostaglandin F derivatives, prostaglandin F_{2α} receptor antagonists, cyclooxygenase-2 inhibitors, muscarinic agents, and combinations thereof.

The approximate solubility in water of some common ophthalmic drugs is shown in Table A.

TABLE A
Solubility of Some Common Ophthalmic Drugs

| | Approximate Solubility |
|---|---|
| Drug Name | in Water (mg/mL) |
| Nonsteroidal Anti-inflammatory Drugs: | |
| indomethacin | 0.01 |
| piroxicam | 0.03 |
| ketoprofen | 0.11 |
| nepafenac | 0.02-0.05 |
| flurbiprofen | 0.008 |
| ketorolac | 0.51⁽*⁾ |
| diclofenac | 0.0008 |
| etodolac | 0.016 |
| suprofen | 0.04⁽*⁾ |
| naproxen | 0.016 |
| bromfenac | 0.013 |
| Immunosupressive Agent: | |
| cyclosporine | 0.0095⁽*⁾ |
| Steroids: | |
| dexamethasone | 0.009 |
| predinisolone | 0.22 |
| betamethasone | 0.0005⁽*⁾ |
| triamcinolone | 0.08 |
| loteprednol etabonate | 0.007⁽*⁾ |
| Anti-infectives: | |
| gramicidin | 0.004⁽*⁾ |
| tetracycline | 0.23 |
| ciprofloxacin | 0.001 |
| moxifloxacin | 0.017⁽*⁾ |
| gatifloxacin | 0.063⁽*⁾ |
| miconazole | 0.0008⁽*⁾ |
| itraconazole | 0.009⁽*⁾ |
| Antiglaucoma Drugs: | |
| epinephrine | 0.18 |
| dipivefrin | 0.058⁽*⁾ |
| brimonidine | 1.5 |
| timolol | 0.27⁽*⁾ |
| betaxolol | 0.45 |
| carteolol | 0.42⁽*⁾ |
| levobutonol | 0.25 |
| demecarium | 0.0001⁽*⁾ |
| dorzolamide | 0.7⁽*⁾ |
| brinzolamide | 0.7⁽*⁾ |
| latanoprost | 0.052 |
| bimatoprost | 0.02⁽*⁾ |
| travoprost | 0.008⁽*⁾ |
| Antihistamines/Mast Cell Stabilizers: | |
| ketotifen | 0.008⁽*⁾ |
| olopatadine | 0.03⁽*⁾ |
| cromoglicate | 0.03⁽*⁾ |

Note:
⁽*⁾denotes a published predicted value.

In one embodiment, the ophthalmic drug having low water solubility included in a composition of the present invention comprises a compound having Formula I or II.

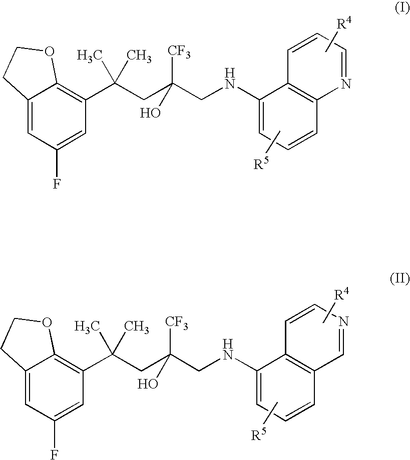

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In another embodiment, the ophthalmic drug having low water solubility comprises a compound having Formula III.

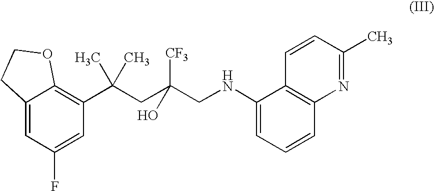

Compound having Formula III has a solubility of about 0.0002 mg/mL in water at pH of about 7.

In still other embodiments, the ophthalmic drug having low water solubility comprises a dissociated glucocorticoid receptor agonist disclosed in published US Patent Application having Ser. No. 11/832294 (Publication No. 2008/0031884), which is incorporated herein by reference in its entirety.

In still another aspect, other drugs that may be formulated in a composition of the present invention include one or more of the following compounds.

Non-limiting examples of the glucocorticosteroids are: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, derivatives thereof, combinations thereof, and mixtures thereof. In one embodiment, the therapeutic agent is selected from the group consisting of difluprednate, loteprednol etabonate, prednisolone, combinations thereof, and mixtures thereof.

Non-limiting examples of the non-steroidal anti-inflammatory drugs (“NSAIDs”) are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

Non-limiting examples of antibiotics include doxorubicin; aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), β-lactams (e.g., carbacephems (e.g., loracarbef)), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Other examples of |antibiotics| are the synthetic antibacterials, such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, gatifloxacin, grepafloxacin, lomefloxacin, miloxacin, moxifloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-B, chloramine-T, dichloramine T, n²-formylsulfisomidine, n⁴-β-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene citrate, methenamine hippurate, methenamine mandelate, methenamine sulfosalicylate, nitroxoline, taurolidine, xibomol).

Non-limiting examples of immunosuppressive agents include dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triaminolone, anecortave acetate, fluorometholone, medrysone, and prednisolone.

Non-limiting examples of antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyirolnitrin, siccanin, tubercidin, viridin, allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiviral agents include acyclovir, carbovir, famciclovir, ganciclovir, penciclovir, and zidovudine.

Non-limiting examples of antiprotozoal agents include pentamidine isethionate, quinine, chloroquine, and mefloquine.

In another aspect, a composition of the present invention comprises: (a) an ophthalmic drug that has a solubility in water in a range from about 0.0001 to about 0.02 mg/mL; and (b) a surfactant, wherein the ophthalmic drug is present at a concentration from about 3 to about 7000 times a solubility of said drug in water, said solubility being measured at about 25° C. and at pH of about 7-7.5, and wherein an amount of said drug is non-toxic to a subject in whom said composition is administered. Alternatively, the ophthalmic drug is present at a concentration from about 10 to about 5000 times a solubility of said drug in water. In some other embodiments, the ophthalmic drug is present at a concentration from about 10 to about 3000 times (or alternatively, from about 10 to about 2000, or from about 10 to about 1000, or from about 10 to about 500, or from about 10 to about 100, or from about 5 to about 100, or from about 5 to about 50, or from about 50 to about 2000, or from about 50 to about 1000, or from about 50 to about 500, or from about 50 to about 100, or from about 100 to about 2000, or from about 100 to about 1000, or from about 100 to about 500 times) a solubility of said drug in water.

Suitable surfactants can include cationic, anionic, non-ionic or amphoteric surfactants. Preferred surfactants are neutral or nonionic surfactants. Non-limiting examples of surfactants suitable for a formulation of the present invention include polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, New York, 2006. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

An ophthalmic composition of the present invention can further comprise one or more other ingredients, such as physiologically acceptable buffers, tonicity adjusting agents, surfactants, viscosity adjusting agents, chelating agents, anti-oxidants, preservatives, or other components.

Non-limiting examples of physiologically acceptable buffers include phosphate buffer; a Tris-HCl buffer (comprising tris(hydroxymethyl)aminomethane and HCl); buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

While the buffer itself is a “tonicity adjusting agent” and a “pH adjusting agent” that broadly maintains the ophthalmic composition at a particular ion concentration and pH, additional “tonicity adjusting agents” can be added to adjust the final tonicity of the composition. Such tonicity adjusting agents are well known to those of skill in the art and include, but are not limited to, mannitol, sorbitol, dextrose, sucrose, urea, propylene glycol, and glycerin. Also, various salts, including halide salts of a monovalent cation (e.g., NaCl or KCl) can be utilized.

The tonicity adjusting agent, when present, can be in a concentration ranging from about 0.01 to about 10, or from about 0.01 to about 7, or from about 0.01 to about 5, or from about 0.1 to about 2, or from about 0.1 to about 1 percent by weight. In some embodiments where a tonicity adjusting agent is present the composition can contain a single agent or a combination of different tonicity adjusting agents. Typically, the tonicity of a formulation of the present invention is in the range from about 200 to 400 mOsm/kg. Alternatively, the tonicity of a formulation of the present invention is in the range from about 220 to 400 mOsm/kg, or from about 220 to 350 mOsm/kg, or from about 220 to 300 mOsm/kg, or from about 250 to 350 mOsm/kg, or from about 280 to 310 mOsm/kg, or from about 290 to 300 mOsm/kg. For relief of dry eye symptoms, an ophthalmic formulation of the present invention may be desirably hypotonic, such as having tonicity in the range from about 200 to about 270 mOsm/kg.

Non-limiting examples of anti-oxidants include ascorbic acid (vitamin C) and its salts and esters; tocopherols (such as α-tocopherol) and tocotrienols (vitamin E), and their salts and esters (such as vitamin E TGPS (D-α-tocopheryl polyethylene glycol 1000 succinate)); glutathione; lipoic acid; uric acid; butylated hydroxyanisole (“BHA”); butylated hydroxytoluene (“BHT”); tertiary butylhydroquinone (“TBHQ”); and polyphenolic anti-oxidants (such as gallic acid, cinnanmic acid, flavonoids, and their salts, esters, and derivatives). In some embodiments, the anti-oxidant comprises ascorbic acid (vitamin C) and its salts and esters; tocopherols (such as α-tocopherol) and tocotrienols (vitamin E), and their salts and esters; or BHA.

In still another embodiment, the amount of an anti-oxidant in a pharmaceutical formulation of the present invention is in the range from about 0.0001 to about 5 percent by weight of the formulation. Alternatively, the amount of an anti-oxidant is in the range from about 0.001 to about 3 percent, or from about 0.001 to about 1 percent, or from greater than about 0.01 to about 2 percent, or from greater than about 0.01 to about 1 percent, or from greater than about 0.01 to about 0.7 percent, or from greater than about 0.01 to about 0.5 percent, or from greater than about 0.01 to about 0.2 percent, or from greater than about 0.01 to about 0.1 percent, or from greater than about 0.01 to about 0.07 percent, or from greater than about 0.01 to about 0.05 percent, or from greater than about 0.05 to about 0.15 percent, or from greater than about 0.03 to about 0.15 percent by weight of the composition, or from greater than about 0.1 to about 1 percent, or from greater than about 0.1 to about 0.7 percent, or from greater than about 0.1 to about 0.5 percent, or from greater than about 0.1 to about 0.2 percent, or from greater than about 0.1 to about 0.15 percent.

In some embodiments, the chelating agent comprises a compound selected from the group consisting of ethylenediaminetetraacetic acid (“EDTA”), diethylenetriaminepentakis (methylphosphonic acid), etidronic acid, pharmaceutically acceptable salts thereof, and mixtures thereof.

In some other embodiments, the chelating agent comprises tetrasodium salt of etidronic acid (also known as “HAP”, which is available as 30% composition).

In still some other embodiments, the chelating agent comprise EDTA sodium salt.

In some embodiments, the ophthalmic compositions of this invention can optionally include one or more viscosity adjusting agents (e.g., particularly when the ophthalmic composition is intended to provide lubrication to an ocular surface (i.e., artificial tear)) or to remain for a longer period on the ocular surface. Suitable viscosity adjusting agents for administration to an eye are well known to those of skill in the art. One or more polysaccharides disclosed above can act as viscosity adjusting agents. Other non-ionic polysaccharides such as cellulose derivatives are commonly used to increase viscosity, and as such, can offer other advantages. Specific cellulose derivatives include, but are not limited to hydroxypropyl methyl cellulose, carboxymethyl cellulose, methyl cellulose, or hydroxyethyl cellulose. Typically, particularly when used as an artificial tear, the ophthalmic composition has a viscosity from about 1 to about 1000 centipoises (or mPa.s). The present composition is usually dispensed in the eye in the form of a suspension. It should be understood, however, that the present composition may also be formulated as a viscous liquid (e.g., viscosities from 50 to several thousand cps), gel, or ointment, which has even higher viscosity, for ophthalmic or, possibly, non-ophthalmic uses.

In some embodiments, an ophthalmic composition of the present invention can further comprise a demulcent. Polysaccharides, such as those disclosed herein above can act as demulcents. Other demulcents also can be included, such as those approved by the U.S. Food and Drug Administration (“US FDA”) and listed in 21 C.F.R. Part 349. They include hypromellose (0.2 to 2.5 percent), dextran 70 (0.1 percent when used with another polymeric demulcent listed in this regulation), gelatin (0.01 percent), liquid polyols, glycerin (0.2 to 1 percent), polyethylene glycol 300 or 400 (0.2 to 1 percent), propylene glycol (0.2 to 1 percent), polyvinyl alcohol (0.1 to 4 percent), povidone (or polyvinyl pyrrolidone, 0.1 to 2 percent). All compositions are in percent by weight of the total formulation, unless otherwise indicated.

In some other embodiments, a composition may include one or more emollients, such as those listed in 21 C.F.R. Section 349.14.

In addition to those classes of ingredients disclosed above, an ophthalmic composition of the present invention can further comprise one or more other ingredients, such as vitamins (other than those disclosed hereinabove), or other ingredients that provide added health benefits to the users.

In another embodiment, a composition of the present invention can further comprise a preservative. In some embodiments, said preservative is polyquaternium-1. In still some embodiments, said preservative is other than a material selected from the group consisting of cationic organic nitrogen-containing compounds and alcohols. In still some other embodiments, said preservative is hydrogen peroxide, urea peroxide, or stabilized oxychloro complex (an equilibrium mixture of oxychloro species). The amount of the preservative is typically less than about 0.1%, or less than about 0.03%, or less than about 0.01% by weight of the total formulation. In still another embodiment, such a preservative is present in an amount from about 0.001 to about 0.01% by weight of the total formulation.

In another aspect, the present invention provides a method for preparing a composition that comprises: (a) an ophthalmic drug that has a low solubility in water; and (b) a surfactant, wherein the ophthalmic drug is present at a concentration from about 3 to about 7000 times a solubility of said drug in water, said solubility being measured at about 25° C. and at pH of about 7-7.5, and wherein an amount of said drug is non-toxic to a subject in whom said composition is administered. The method comprises: (a) adding a predetermined amount of the drug to a predetermined amount of said surfactant and an amount of a ophthalmically acceptable carrier, wherein the drug is in a form of particles less than about 1 μm (alternatively, less than about 0.5 μm, or less than about 0.2 μm), and wherein the amount of carrier is sufficient to produce the desired final concentration of the drug; and (b) mixing the drug, the surfactant, and the carrier together to produce the composition.

In one aspect, the composition is a suspension.

In another aspect, a composition of the present invention can be prepared by a method comprising the step of, (a) adding a predetermined amount of a surfactant into a vessel containing 80-90 percent of a desired amount of the carrier; (b) adding a predetermined amount of the drug, into the vessel; (c) mixing the contents of the vessel for a time to yield a substantially uniform mixture; (d) adding another amount of the carrier to the vessel to bring the total volume of the mixture to 100 percent of the desired volume; and (e) mixing the contents of the vessel further to produce the final composition. The method can further comprise subjecting the composition to sterilization by heating, autoclaving and/or filtration through a desired filter. Optionally, the method also can comprise adding desired amounts of one or more additional ingredients to the vessel, which additional ingredients are selected from the group consisting of buffers, tonicity adjusting agents, chelating agents, demulcents, emollients, viscosity adjusting agents, other vitamins, other ingredients that provide added health benefits to the users, and mixtures thereof

### EXAMPLE 1

### Increasing Dose Volume at Constant Drug Concentration

This study shows that increasing the dose volume at a constant drug concentration yields less than proportional increase in the bioavailability of the drug in ocular tissues following topical administration of the composition.

An aqueous suspension comprising 0.1 mg/mL of the compound having Formula III (BOL-303242-X), 100 mg/mL PEG 3350, and 10 mg/mL Tween® 80 was prepared substantially as disclosed above.

This study was carried out using a non-crossover design and a total of 84 naïve pigmented male rabbits (Dutch Belted). Each rabbit was arbitrarily assigned to one of three dose groups.

On the day of dosing, each animal received a topical ocular dose of the suspension. Animals in Group 1 received a 10-μL instillation, Group 2 received a 30-μL instillation and Group 3 received a 100-μL instillation. The target dose levels for these groups were 1, 3 and 10 μg/eye, respectively. The test composition was shipped to the test site as a ready-to-use suspension.

At predetermined time intervals after dosing, rabbits (four per collection time) were euthanized and the eyes enucleated and dissected. Samples of tear, conjunctiva, cornea, aqueous humor and iris/ciliary body were obtained from each animal and frozen until being shipped on dry ice to the analytical laboratory. Samples were collected at 0.0833, 0.25, 0.5, 1, 2, 4 and 6 hour after dosing. Concentrations of BOL-303242-X in ocular tissue samples were determined by LC/MS/MS. One or more samples of each tissue had concentrations that were below the lower limit of quantitation (“LLQ”). These samples were assigned a value of 0.5 times the LLQ for the purpose of calculating a mean concentration for pharmacokinetic (“PK”) analysis. In addition, the BOL-303242-X concentration measured in one tear sample collected at 6 hour was more than 100-fold higher than any other 6-hour sample for the same dose group. This value was considered to be an outlier, and was not included in any calculations. Non-compartmental methods were used for pharmacokinetic analysis of concentration versus time data (WinNonlin version 5.2). Due to the destructive sampling regimen employed in this study average composite data were used in the PK analysis.

Results: The following pharmacokinetic parameters were obtained.

TABLE 1

| | | | | | | DNAUC |
|---|---|---|---|---|---|---|
| | Dose Volume | Dose | Cmax | Tmax | AUC(0-t) | (ng * hr/g ÷ |
| Tissue | (μL) | (μg) | (ng/g) | (hr) | (ng * hr/g) | μg dose) |
| Tear | 10 | 1 | 16300 ± 7000 | 0.083 | 8870 | 8870 |
| | 30 | 3 | 28100 ± 11300 | 0.25 | 12600 | 4200 |
| | 100 | 10 | 36700 ± 21400 | 0.25 | 25600 | 2560 |
| Aqueous | 10 | 1 | <1 | Not Reported^{(a)} | | |
| Humor | 30 | 3 | <1 | | | |
| | 100 | 10 | <1 | | | |
| Conjunctiva | 10 | 1 | 292 ± 137 | 0.083 | 245 | 245 |
| | 30 | 3 | 684 ± 446 | 0.083 | 487 | 162 |
| | 100 | 10 | 1050 ± 531 | 0.083 | 741 | 74.1 |
| Cornea | 10 | 1 | 288 ± 116 | 0.25 | 270 | 270 |
| | 30 | 3 | 716 ± 234 | 0.083 | 634 | 211 |
| | 100 | 10 | 955 ± 619 | 0.083 | 955 | 95.5 |
| Iris/Ciliary | 10 | 1 | 19.6 ± 29.8 | 1.0 | 32.0 | 32.0 |
| Body | 30 | 3 | 13.1 ± 19.0 | 2.0 | 33.8 | 11.3 |
| | 100 | 10 | 11.3 ± 8.05 | 1.0 | 30.5 | 3.05 |

Abbreviations: Cmax denotes Maximum mean (± standard deviation) concentration observed after dosing; Tmax denotes time at which Cmax was observed; AUC(0-t) denotes area under the mean concentration versus time curve from time zero to the time (t) of the last measurable concentration; DNAUC denotes dose-normalized AUC, calculated as the AUC(0-t) divided by the dose (in μg).
Note:
For aqueous humor, relevant units for Cmax are ng/mL.
^{(a)}The BOL-303242-X concentration in most aqueous humor samples was below the lower limit of quantitation (“BLQ”); no PK analysis was performed.

### Summary of Findings:

Despite the large inter-animal variability, there was a consistent trend observed for BOL-303242-X concentrations in tear, cornea and conjunctiva to increase with increasing instillation volume (and thus dose) in the present study. However, any increase in exposure was less than proportional to the administered dose/volume. BOL-303242-X concentrations in aqueous humor and iris/ciliary body appeared to be similar for each of the dose groups.

### EXAMPLE 2

### Decreasing Dose Volume While Keeping the Same Total Amount of Drug

This study shows that decreasing the dose volume while keeping the same total amount of drug administered increases the bioavailability of the drug in the ocular tissues.

This study was carried out using a non-crossover design involving a total of 84 New Zealand Composite pigmented rabbits arbitrarily divided into 3 groups of 28 rabbits each. Three compositions were prepared as aqueous suspensions substantially as disclosed above comprising 1, 0.33, and 0.1 mg/mL BOL-303242-X, respectively. Each composition also contained 100 mg/mL PEG 3350 and 10 mg/mL Tween® 80. The compositions were shipped to the test site as a ready-to-use suspension.

The test compositions were administered so as to achieve 10-μg topical dosed drug amount in each eye of all the study animals. The concentration of the composition and the instillation volume were varied between the three study groups to keep the dosed drug amount constant. Animals in Group 1 received 10 μL/eye of the 1.0 mg/mL composition; animals comprising Groups 2 and 3 received 30 μL/eye of the 0.33 mg/mL composition and 100 EL/eye of the 0.1 mg/mL composition, respectively.

At predetermined time intervals after dosing, rabbits (four per collection time) were euthanized after anesthetization and the eyes enucleated and dissected. Tear, aqueous humor, conjunctiva, cornea, and iris/ciliary body samples were collected from both eyes of each animal and stored frozen until being shipped on dry ice to the analytical laboratory. Samples were collected at 0.083, 0.25, 0.5, 1, 2, 4, and 6 hours after dosing. BOL-303242-X concentrations in the above tissues and matrices were determined by LC/MS/MS. For the purpose of calculating mean concentrations, a value of ½ the LLQ was assigned to all samples with concentrations below the LLQ (BLQ). In addition, any sample with a measured concentration that was more than 10-fold higher than the median concentration in the respective sample pool was considered an outlier, and was not included in any calculations. Similarly, concentrations that were 10-fold lower than the median value and below the LLQ were also considered to be outliers. The number of study samples that were determined to be outliers using the above criteria for each tissue/matrix in this study was: tears=14/144; conjunctiva=3/144; cornea=1/144; aqueous humor 2/144 and iris=4/144 samples.

Pharmacokinetic analysis of the composite concentration-versus-time data was performed using non-compartmental methods (WinNonlin version 5.2). Due to the destructive nature of the sampling regimen employed in this study, average composite data were used in the pharmacokinetic analysis.

Results: The following pharmacokinetic parameter values were obtained.

TABLE 2

| | | | | | DNAUC |
|---|---|---|---|---|---|
| Instillation | | Cmax | Tmax | AUC(0-t) | (ng * hr/g ÷ |
| Volume | Tissue/Matrix | (ng/g) | (hr) | (μg * hr/g) | μg dose) |
| 10 μL/eye | Tears | 247000 ± 258000 | 0.25 | 60.2 | 6020 |
| (1 mg/mL) | Conjunctiva | 399 ± 100 | 0.083 | 0.742 | 74.2 |
| | Cornea | 527 ± 213 | 0.5 | 1.01 | 101 |
| | Aqueous Humor | 0.170 ± 0.110 | 2.0 | 0.00800 | 0.800 |
| | Iris/Ciliary Body | 262 ± 295^{(a)} | 2.0 | 0.273 | 27.3 |
| 30 μL/eye | Tears | 32000 ± 15500 | 0.083 | 18.2 | 1820 |
| (0.33 mg/mL) | Conjunctiva | 450 ± 269 | 0.25 | 0.575 | 57.5 |
| | Cornea | 592 ± 297 | 0.083 | 1.02 | 102 |
| | Aqueous Humor | 0.188 ± 0.150 | 2.0 | 0.00300 | 0.300 |
| | Iris/Ciliary Body | 41.9 ± 37.8 | 0.083 | 0.0206 | 2.06 |
| 100 μL/eye | Tears | 7170 ± 5000 | 0.25 | 7.74 | 774 |
| (0.1 mg/mL) | Conjunctiva | 168 ± 101 | 0.25 | 0.212 | 21.2 |
| | Cornea | 452 ± 129 | 0.25 | 0.745 | 74.5 |
| | Aqueous Humor | 0.286 ± 0.184 | 2.0 | 0.00700 | 0.700 |
| | Iris/Ciliary Body | 5.92 ± 7.26 | 1.0 | 0.0186 | 1.86 |

Abbreviations: Cmax denotes maximum mean (± standard deviation) concentration observed after dosing; Tmax denotes time Cmax was observed; AUC(0-t) denotes area under the concentration versus time curve from the time of dosing through time (t) of the last measurable concentration; DNAUC denotes dose-normalized AUC, calculated as AUC(0-t) (in ng * hr/g units) divided by the dose (in μg).
Note:
For aqueous humor the relevant units for Cmax and AUC are ng/mL and μg * hr/mL, respectively.
^{(a)}Cmax and AUC in iris/ciliary body were heavily influenced by the high concentrations observed in 2 animals in the 2-hour sampling group.

### Summary of the Findings

The results from this study indicate that ocular |bioavailability| of BOL-303242-X increases when the dose volume is decreased while the drug concentration in the formulation is increased proportionally. Better ocular bioavailability was achieved by this method than by increasing the dose volume alone, as shown by a comparison of the results of the two studies.

Based on the absolute Cmax and AUC values, the exposure to BOL-303242-X in tears increases as the concentration of the drug in the dose volume increases. A similar, though less pronounced trend is observed for the conjunctiva.

In contrast, based on the absolute Cmax and AUC values, exposure to BOL-303242-X in cornea, iris/ciliary body and aqueous humor remained relatively constant regardless of the changes in the instillation volume/concentration.

Thus, in one aspect, a composition or a method of the present invention can provide at least one of the following advantages: provision of greater efficiency in delivering the drug to a target tissue (i.e., greater dose-normalized AUC) and avoidance of lower absolute concentrations/Cmax/AUC (thus, obtaining the benefit of maintaining comparable therapeutic efficacy with less drug).

In another aspect, a composition or a method of the present invention can bring a benefit of minimizing the effect of the vehicle. For example, a low dose volume can help to detect more easily the difference between the drug and vehicle or can minimize any undesired effect of a preservative in the composition.

In still another aspect, an ophthalmic composition of the present invention comprising a desired ophthalmic active ingredient can be used to treat ocular conditions such as dry eye, inflammation, allergy, or infection of the eye.

In still another aspect, the present invention provides methods of making and using compositions of the present invention. Any of the materials, compounds, and ingredients disclosed herein is applicable for use with, or inclusion in, any method of the present invention.

In yet another aspect, the present invention provides a method for increasing bioavailability of an ophthalmic drug that has a low solubility in water, in an ocular of a subject. The method comprises administering topically to an ocular surface of said subject a volume from about 1 to about 15 microliter (“μL”) of a composition that comprises the ophthalmic drug, wherein the ophthalmic drug is present at a concentration from about 3 to about 7000 times a solubility of said drug in water, said solubility being measured at about 25° C. and at pH of about 7-7.5, and wherein an amount of said drug is non-toxic to said subject.

Alternatively, the concentration of the ophthalmic drug can be in a range selected from those disclosed herein above.

In a further aspect, the ophthalmic drug included in a composition, and used in a method, of the present invention comprises a compound having Formula I, II, or III and is effective for increasing its bioavailability for the treatment, control, or prevention of ocular inflammation or dry eye.

In one embodiment, such ocular inflammation comprises anterior segment inflammation.

In yet another aspect, the present invention provides a method for treating, controlling, or preventing a condition of an eye with an ophthalmic active ingredient that has a low solubility in water. The method comprises administering topically to an ocular surface of a subject a volume from about 1 to about 15 microliter (“μL”) of a composition that comprises the ophthalmic active ingredient, wherein the ophthalmic active ingredient is present at a concentration from about 3 to about 7000 times the solubility of said active ingredient in water, said solubility being measured at about 250 C and at pH of about 7-7.5, and wherein the amount of said active ingredient in said volume is non-toxic to said subject.

Alternatively, the concentration of the ophthalmic drug can be in a range selected from those disclosed herein above. For example, the ophthalmic drug is present at a concentration from about 10 to about 5000 times a solubility of said drug in water. In some other embodiments, the ophthalmic drug is present at a concentration from about 10 to about 3000 times (or alternatively, from about 10 to about 2000, or from about 10 to about 1000, or from about 10 to about 500, or from about 10 to about 100, or from about 5 to about 100, or from about 5 to about 50, or from about 50 to about 2000, or from about 50 to about 1000, or from about 50 to about 500, or from about 50 to about 100, or from about 100 to about 2000, or from about 100 to about 1000, or from about 100 to about 500 times) a solubility of said drug in water.

In still another aspect, a composition of the present invention is instilled into an affected eye at a dosage of one, two, three, four, or more drops per day, or as prescribed by a skilled medical practitioner, wherein each drop has a volume from about I to about 15 μL. For example, one, two, or three drops of a formulation of the present invention are instilled into an affected eye once, twice, three or more times per day. In certain embodiments, the volume of a drop is about 10-15 μL.

The following non-limiting examples show other compositions of the present invention.

### EXAMPLE 3:

TABLE 3

| | Amount |
|---|---|
| Ingredient | (percent w/v) |
| Polyhexamethylenebiguanide HCl (as a 20 percent | 0.00047 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Poloxamine (Tetronic ® 1107 from BASF) | 1.00 |
| Tetrasodium etidronate (as a 30 percent w/w solution, | 0.01 |
| available under the mark DeQuest ® 2016 | |
| from Monsanto Co.) | |
| Indomethacin | 1 |
| Hydrochloric acid (1N) or sodium hydroxide (1N) | as required to |
| | adjust pH to 7-7.5 |
| Purified water | q.s. to 100 |

### EXAMPLE 4:

TABLE 4

| | Amount |
|---|---|
| Ingredient | (percent w/v) |
| Polyhexamethylenebiguanide HCl (as a 20 percent | 0.00047 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Flurbiprofen | 3 |
| Hydrochloric acid (1N) or sodium hydroxide (1N) | as required to |
| | adjust pH to 7-7.5 |
| Purified water | q.s. to 100 |

### EXAMPLE 5:

TABLE 5

| | Amount |
|---|---|
| Ingredient | (percent w/v) |
| Polyhexamethylenebiguanide HCl (as a 20 percent w/w | 0.0006 |
| solution available under the mark Cosmocil CQ, from ICI | |
| Chemical Co.) | |
| Phosphate buffer | 0.5 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 from Monsanto | |
| Co.) | |
| Dexamethasone | 1 |
| Purified water | q.s. to 100 |

### EXAMPLE 6:

TABLE 6

| | Amount |
|---|---|
| Ingredient | (percent w/v) |
| Polyhexamethylenebiguanide HCl (as a 20 percent w/w | 0.0005 |
| solution available under the mark Cosmocil CQ, from ICI | |
| Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Ciprofloxacin | 2 |
| Hydrochloric acid (1N) or sodium hydroxide (1N) | as required to |
| | adjust pH to |
| | 7-7.5 |
| Purified water | q.s. to 100 |

### EXAMPLE 7:

TABLE 7

| | Amount |
|---|---|
| Ingredient | (percent w/v) |
| Polyhexamethylenebiguanide HCl (as a 20 percent w/w | 0.0005 |
| solution available under the mark Cosmocil CQ, from ICI | |
| Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 from | |
| Monsanto Co.) | |
| Latanoprost | 2 |
| Hydrochloric acid (1N) or sodium hydroxide (1N) | as required to |
| | adjust pH to |
| | 7-7.5 |
| Purified water | q.s. to 100 |

### EXAMPLE 8:

TABLE 8

| | Amount |
|---|---|
| Ingredient | (percent w/v) |
| Stabilized chlorine dioxide complex | 0.01 |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 from | |
| Monsanto Co.) | |
| Ketotifen | 2 |
| Olopatadine | 1 |
| Hydrochloric acid (1N) or sodium hydroxide (1N) | as required to |
| | adjust pH to 7-7.5 |
| Purified water | q.s. to 100 |

### EXAMPLE 9:

TABLE 9

| Ingredient | Amount (percent w/v) |
|---|---|
| Benzalkonium chloride | 0.0005 |
| Boric acid | 0.64 |
| Sodium borate | 0.12 |
| Edetate disodium | 0.11 |
| Sodium chloride | 0.49 |
| Polysorbate 80 | 0.7 |
| Cyclosporine | 5 |
| Hydrochloric acid (1N) or sodium hydroxide (1N) | as required to |
| | adjust pH to 7-7.5 |
| Purified water | q.s. to 100 |

### EXAMPLE 10:

TABLE 10

| | Amount |
|---|---|
| Ingredient | (percent w/v) |
| Polyquaternium-1 | 0.01 |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 from | |
| Monsanto Co.) | |
| Dipivefrin | 5 |
| Hydrochloric acid (1N) or sodium hydroxide (1N) | as required to |
| | adjust pH to 7-7.5 |
| Purified water | q.s. to 100 |

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. An ophthalmic composition comprising an ophthalmic drug that has a low solubility in water and a surfactant, wherein the ophthalmic drug is present at a concentration from about 3 to about 7000 times the solubility of said drug in water, said solubility being measured at about 25° C. and at pH of about 7-7.5.

2. The composition of claim 1, wherein the concentration is in a range from about 10 to about 1000 times the solubility of said drug in water, and the solubility is in a range from about 0.0001 to about 0.05 mg/mL.

3. The composition of claim 1, wherein the concentration is in a range from about 100 to about 500 times the solubility of said drug in water, and the solubility is in a range from about 0.0001 to about 0.05 mg/mL.

4. The composition of claim 1, wherein the ophthalmic drug comprises a compound having Formula I or II 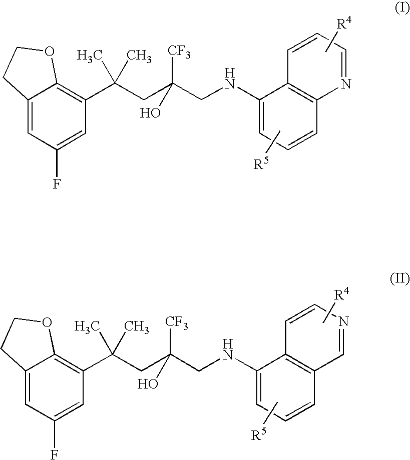
wherein R⁴ and R⁵ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

5. The composition of claim 1, wherein the ophthalmic drug comprises a compound having Formula III

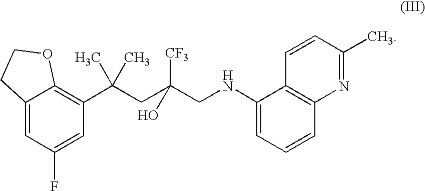

6. The composition of claim 1, wherein the ophthalmic drug is selected from the group consisting of anti-inflammatory agents, anti-infective agents, anti-allergic agents, antihistamines, antiproliferative agents, anti-angiogenic agents, anti-oxidants, antihypertensive agents, neuroprotective agents, cell receptor agonists, cell receptor antagonists, immunomodulating agents, immunosuppressive agents, intraocular lowering agents, α₂-adrenergic receptor agonists, β₁-adrenergic receptor antagonists, carbonic anhydrase inhibitors, cholinesterase inhibitor miotics, prostaglandins and prostaglandin receptor agonists, mast cell degranulation inhibitors (mast cell stabilizers), thromboxane A₂ mimetics, protein kinase inhibitors, prostaglandin F derivatives, prostaglandin F_{2α} receptor antagonists, cyclooxygenase-2 inhibitors, muscarinic agents, and combinations thereof.

7. The composition of claim 6, wherein the composition is an aqueous suspension.

8. The composition of claim 7, wherein the ophthalmic drug is in the form of particles having a size less than about 1 micrometer.

9. A method for preparing an ophthalmic composition, the method comprising: (a) adding a predetermined amount of an ophthalmic drug to a predetermined amount of a surfactant and an amount of an ophthalmically acceptable carrier; and (b) mixing the drug, the surfactant, and the carrier together to produce the composition, wherein the ophthalmic drug has a low solubility in water, the drug is in a form of particles less than about 1 μm, the amount of the carrier is sufficient to produce a desired final concentration of the drug, the ophthalmic drug is present at a concentration from about 3 to about 7000 times a solubility of the drug in water, the solubility being measured at about 25° C. and at pH of about 7-7.5, and the amount of the drug in a volume administered to a subject is non-toxic to the subject.

10. A method for treating, controlling, or preventing a condition of an eye of a subject with an ophthalmic active ingredient, the method comprising administering topically to an ocular surface of the subject a volume from about 1 to about 15 microliter of a composition that comprises the ophthalmic active ingredient, wherein the ophthalmic active ingredient has a low solubility in water and is present at a concentration from about 3 to about 7000 times the solubility of said active ingredient in water, said solubility being measured at about 25° C. and at pH of about 7-7.5, and the amount of said drug in said volume is non-toxic to said subject.

11. The method of claim 10, wherein the concentration is in a range from about 10 to about 1000 times the solubility of said drug in water, and the solubility is in a range from about 0.0001 to about 0.05 mg/mL.

12. The method of claim 10, wherein the concentration is in a range from about 100 to about 500 times the solubility of said drug in water, and the solubility is in a range from about 0.0001 to about 0.05 mg/mL.

13. The method of claim 10, wherein the ophthalmic active ingredient comprises a compound having Formula I or II 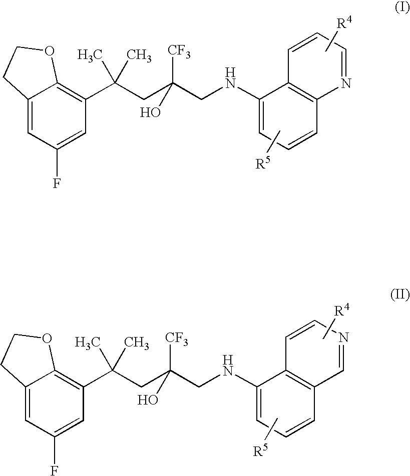
wherein R⁴ and R⁵ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

14. The method of claim 10, wherein the ophthalmic active ingredient comprises a compound having Formula III

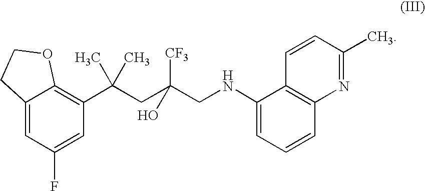

15. The method of claim 14, wherein the condition comprises ocular inflammation or dry eye.

16. The method of claim 10, wherein the ophthalmic active ingredient is selected from the group consisting of anti-inflammatory agents, anti-infective agents, anti-allergic agents, antihistamines, antiproliferative agents, anti-angiogenic agents, anti-oxidants, antihypertensive agents, neuroprotective agents, cell receptor agonists, cell receptor antagonists, immunomodulating agents, immunosuppressive agents, intraocular lowering agents, α₂-adrenergic receptor agonists, β₁-adrenergic receptor antagonists, carbonic anhydrase inhibitors, cholinesterase inhibitor miotics, prostaglandins and prostaglandin receptor agonists, mast cell degranulation inhibitors (mast cell stabilizers), thromboxane A₂ mimetics, protein kinase inhibitors, prostaglandin F derivatives, prostaglandin F_{2α} receptor antagonists, cyclooxygenase-2 inhibitors, muscarinic agents, and combinations thereof.

17. The method of claim 16, wherein the composition is an aqueous suspension.

18. The method of claim 17, wherein the volume of the composition administered to the ocular surface is from about 5 to about 15 microliter.

19. The method of claim 10, wherein the volume of the composition administered to the ocular surface is from about 5 to about 15 microliter.

20. The method of claim 14, wherein the volume of the composition administered to the ocular surface is from about 5 to about 15 microliter.
